(19) United States
(12) Patent Application Publication
(10) Pub. No.: US 2005/0171425 A1
(43) Pub. Date: Aug. 4, 2005

(54) Medical devices having MRI-enhancing encapsulated fluids

(75) Inventor: Thomas M. Burke, Bothell, WA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: Phantoms-By-Design, Bothell, WA (US)

(21) Appl. No.: 10/759,908

(22) Filed: Jan. 16, 2004

(51) Int. Cl. A61B 5/05

(52) U.S. Cl. 600/420; 600/410; 600/411

(57) ABSTRACT

A medical device is described that comprises a polymeric component and a microcapsule additive including a fluid that enhances direct visibility during magnetic resonance imaging of at least a predetermined portion of the polymeric component.

### FIELD OF THE INVENTION

The present invention relates to medical devices and, more particularly, to interventional medical devices that include microencapsulated fluids to enhance MRI visibility.

### BACKGROUND OF THE INVENTION

Cardiovascular disease is currently one of the world's biggest health care issues, and it is the leading cause of death in many countries including the United States. Despite a variety of clinical presentations, the majority of cardiovascular diseases are related to atherosclerosis. Emerging research has identified pathogenic attributes associated with disease progression. Some of these attributes include subtle vessel wall features linked to unstable plaque as well as microvascular dysfunction linked to angina and ischemia.

New techniques offer the promise of early detection with minimally invasive treatment prior to acute clinical syndromes, arterial occlusions and infarction. To maximize clinical utility, diagnosis, risk stratification, it would be desirable to consolidate intervention and subsequent treatment using a single modality.

Magnetic Resonance Imaging (MRI) advantageously permits precise imaging during treatment, particularly with the recent availability of gadolinium enhanced, three-dimensional, gradient echo imaging techniques. Because current standards of care call for minimally invasive treatment early in the disease process, MRI may become a valuable modality for diagnosing and treating cardiovascular disease.

Image-guided intervention spans multiple procedures (e.g., biopsy, stent placement, arterial embolization, RF ablation), and typically involve guiding an interventional device to an examination or treatment area while minimizing damage to adjacent healthy tissue. Interventional device guidance generally is performed in real time using two-dimensional imagery, and multiple images from different perspectives may be required to guide the interventional device through complex anatomy. Once the interventional device is positioned, diagnostic quality three-dimensional images are acquired to insure proper placement and post deployment functionality.

Magnetic resonance image-guided intervention generally requires high spatial (e.g., ˜1 mm) and temporal (e.g., 3-20 frames per second) resolution with good signal strength (e.g., signal-to-noise ratio >10) and contrast (e.g., contrast-to-noise ration >7) between the interventional devices and the patient's tissue. In addition, the interventional device should be directly visible, independent of the orientation at which the image is obtained, and without resorting to specialized image sequences, hardware or post processing that may reduce real-time visualization. Moreover, the interventional device preferably is safe for the patient and operator, should not produce image artifacts, should be stable after long storage, should maintain performance during extended procedures, and should be compatible with existing materials and methods.

A number of techniques currently are under development to provide tracking of interventional devices under MRI guidance. Such techniques may be categorized as either passive or active. Passive tracking systems include both bright and dark instrument enhancements, wherein bright enhancements may be generated by filling the catheter lumen with T1 shortening agents (e.g., T1˜360 ms, T2˜280 ms). Dark enhancements (or signal voids) may be generated when a hypo-intensive instrument displaces tissue or contrast-enhanced blood pools.

Active tracking systems generally are used as high contrast beacons to localize the tip of an interventional device, i.e., a catheter, in real time at high frame rates. The tip location is displayed as an overlay on a previously acquired anatomic roadmap image. Active systems may be grouped into those that: (1) locally control signal strength; (2) operate strictly as a location beacon, (3) allow visualization of a length of receiver coil and facilitate local imaging; and (4) combine more than one of features (1) to (3).

Despite the promise offered by MRI, there exist technical challenges to successful widespread adoption of this technique. For example, MRI is a relatively slow imaging technique, and diagnostic procedures suffer from image artifacts caused by tissue motion occurring during the extended periods required for image formation. Moreover, most interventional devices are not compatible with MRI, either because the devices incorporate metal components, or are manufactured from polymers that are not visible in magnetic resonance images.

For example, most polymers used in catheter construction are not visible under MRI because they lack free protons. While it would be desirable to incorporate fluids into the polymer matrix of interventional devices to enhance visibility, the presence of fluids may either inhibit polymerization of the polymer matrix of the interventional device, or lead to materials having poor mechanical properties.

Previous efforts to overcome problems associated with passive visualization of interventional devices in MRI images have focused on incorporating paramagnetic materials into the body of the interventional devices, such as catheters. For example, U.S. Pat. No. 5,728,079 to Weber et al. and U.S. Pat. No. 5,817,017 to Unger et al. describe catheters having enough paramagnetic material to render at least a portion of the catheter body visible under MRI. A drawback of such devices, however, is that the mechanical properties of the catheters are expected to vary from catheters manufactured from more conventional materials, thus hindering adoption of MRI techniques.

Other previously known attempts to enhance visualization of internal body structures, e.g., for diagnostic imaging, have been directed to the use contrast agents comprising gel particles that encase a metallic material, as described, for example, in U.S. Pat. No. 5,976,500 to Unger. U.S. Pat. No. 5,368,840 describes the use of natural polymers as contrast agents for use in MRI diagnostic imaging procedures. While such contrast agents of the foregoing patents may be safe and effective for introduction into a patient's circulatory system, it is expected that such contrast agents could not withstand the manufacturing processes needed to incorporate into interventional devices to enhance the visibility of such devices under MRI.

Microparticles and nanoparticles also have been developed for use with MRI, primarily as tissue specific and blood pool contrast agents. Iron oxide nanoparticles with oxide starch coatings are not directly visible, but affect MRI visibility through enhancement of local magnetic fields for specific tissue types. However, these nanoparticles remain in the patient's blood pool for extended periods of time.

Microparticles comprising gadolinium or gold impregnated polyvinyl alcohol or tris acrylimide gel spheres (e.g., 300-710 microns) have been used for direct MRI visualization of therapeutic arterial embolization. Further, embolic microparticles have been used for therapeutic arterial occlusion and end organ infarction. However, most existing microparticle contrast agents are not directly visible in MRI and suffer from poor signal strength and tissue contrast. Additionally, most microparticle contrast agents are not compatible with commonly-used medical grade polymers and solvents.

In view of the aforementioned drawbacks, it would be desirable to provide medical devices, including interventional devices, that comprise materials that enhance visibility under MRI, which are biocompatible, and that are sufficiently robust to withstand the manufacturing processes typically encountered in medical device construction.

It further would be desirable to provide materials that may be incorporated into medical devices, including interventional devices, with enhanced visibility under MRI, but which do not substantially impact the mechanical properties of the devices.

It also would be desirable to provide materials that may be incorporated into medical devices, such as interventional devices and contrast agents, with enhanced visibility under MRI, wherein the materials comprise fluid encapsulated in thin, environmentally robust shells capable of withstanding manufacturing processes employed with medical grade polymers and/or environmental conditions encountered in the human body.

It further would be desirable to provide a stable, biocompatible contrast additive for use with medical grade polymers that allows medical devices, such as catheters and contrast agents, to be directly visualized under clinical MRI conditions.

### SUMMARY OF THE INVENTION

In view of the foregoing, it is an object of the present invention to provide medical devices, including interventional devices, that comprise materials that enhance visibility under MRI, which are biocompatible, and that are sufficiently robust to withstand the manufacturing processes typically encountered in device construction.

It is another object of this invention to provide materials that may be incorporated into medical devices, including interventional devices, with enhanced visibility under MRI, but which do not substantially impact the mechanical properties of the devices.

It also is an object of the present invention to provide materials that may be incorporated into medical devices, such as interventional devices and contrast agents, with enhanced visibility under MRI, wherein the materials comprise fluid encapsulated in thin, environmentally robust shells capable of withstanding manufacturing processes employed with medical grade polymers and environmental conditions encountered in the human body.

It is a further object of this invention to provide a stable, biocompatible contrast additive for use with medical grade polymers that allows medical devices, such as catheters, to be directly visualized under clinical MRI conditions.

In accordance with the principles of the present invention, a medical device, such as a catheter, comprises a device body including a distal end, wherein at least a portion of the medical device comprises a mixture of polymeric material and microcapsules containing fluids that render at least a portion of the device body visible under MRI.

In one embodiment of the present invention, the microcapsules comprise thin, environmentally robust shells formed by coacervation that contain lipids or oils, such as mineral oil, cod liver oil, terpene or polyunsaturated fatty acids. The fluids contained within the microcapsules provide a source of free protons to enhance visibility under MRI, while the shells of the microcapsules physically isolate the fluid from the polymer matrix in which the microcapsules are incorporated. According to some embodiments, the device body may further comprise relaxation modifying agents, such as iron oxide particles or anchored gadolinium based T1 reducing agents, that limit fluid mobility.

Further in accordance with the principles of the present invention, neither the shells nor cores of the microcapsules have air inclusions that could reduce the population of available protons and/or produce susceptibility artifacts in the MR images. In addition, the shells of the microcapsules preferably comprise materials that provide stability, and the ability to withstand prolonged exposure to organic solvents, high temperatures and high pressures.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout.

FIG. 1 is a photograph of microcapsules of the present invention formed using coacervation techniques that are per se known in the art of microencapsulation;

FIGS. 2A-2C are side views of illustrative catheters constructed in accordance with the principles of the present invention to include microencapsulated fluids; and

FIG. 3 is a collage of magnetic resonance images under varied pulse sequences that compare the visibility of a test sample constructed of standard polyurethane under MRI with a test sample constructed in accordance with the principles of the present invention.

### DETAILED DESCRIPTION OF THE INVENTION

The present invention is directed to medical devices having enhanced visibility under MRI, wherein microcapsules containing MRI contrast additives are dispersed in at least a portion of the device comprising medical grade polymer matrices. The microcapsules allow the construction of MRI compliant medical devices that exhibit clinically relevant MRI visibility while retaining favorable mechanical and manufacturing properties. The present invention may be especially advantageous for construction of diagnostic and therapeutic interventional devices, such as catheters. The contrast additive of the present invention is compatible with materials and manufacturing methods currently used to make medical devices, is patient-safe, and produces clinically acceptable visibility independent of viewing angle or pulse sequence.

While the present invention is illustratively described in the context of catheters suitable for use in MRI-guided diagnostic or treatment procedures, it will be understood that the microcapsules may be incorporated in any of a variety of types of medical devices. For example, the microcapsules of the present invention may advantageously be incorporated into the matrix of, or a polymer coating of, medical devices, such as interventional devices, for diagnosis or treatment of cardiovascular defects (such as catheters, stents, stent delivery systems and contrast agents), stenoses, treatment of gastrointestinal ailments, etc.

The microcapsules of the present invention also are expected to be advantageous with respect to:
external fiducial markers that may be placed on a patient to assist in registering patients to high resolution pre-intervention images;
embedded fiducial markers for navigation in a compliant tissue environments;
embedded high contrast targets for use in oncology treatments;
directly visible interventional devices to assist in localizing interventional devices within pre-interventional images;
MRI visible, biocompatible implants (grafts, valves, stents, etc.); and
MRI visible, biocompatible aneurysm coils.

Most polymers used in medical devices, such as interventional devices, are hypo-intensive because they lack a large population of free protons in the polymer matrix with sufficiently long T2 times (typically ˜2 ms). The microcapsules of the present invention overcome this deficiency by adding fluids that are physically isolated from the monomers during matrix formation, thereby increasing the available pool of free protons with long T2 times. The microcapsules of the present invention thus provide a way to introduce isolated fluids into a portion of the polymer matrix of a medical device while preserving the strength and elasticity of the polymer matrix.

According to a preferred embodiment, the microcapsules of the present invention comprise a central fluid reservoir disposed within a thin, environmentally robust shell. The shell and fluid reservoir preferably are substantially free of air inclusions, which might otherwise reduce the available population of free protons and produces susceptibility artifacts in the magnetic resonance images. The shells of the microcapsules preferably provide stability, a long shelf-life, and the ability to withstand prolonged exposure to hostile environmental conditions including, but not limited to, organic solvents (e.g., tetrahydrofuran), high temperatures and high pressures.

In a preferred embodiment of the present invention, a medical device, such as an MRI catheter, is formed by mixing microcapsules with medical grade polymers (e.g., polyurethane), and then casting or extruding the catheter using conventional manufacturing processes. The included microcapsules enhance visibility of the catheter in magnetic resonance images. As described hereinbelow, the microcapsules of the present invention permit passive visualization of the interventional device by enhancing the brightness of the device in the MRI image. More particularly, the microcapsules of the present invention permit a medical device, such as an interventional catheter, to be made with a significantly smaller wall thickness, while maintaining or enhancing visualization of the device.

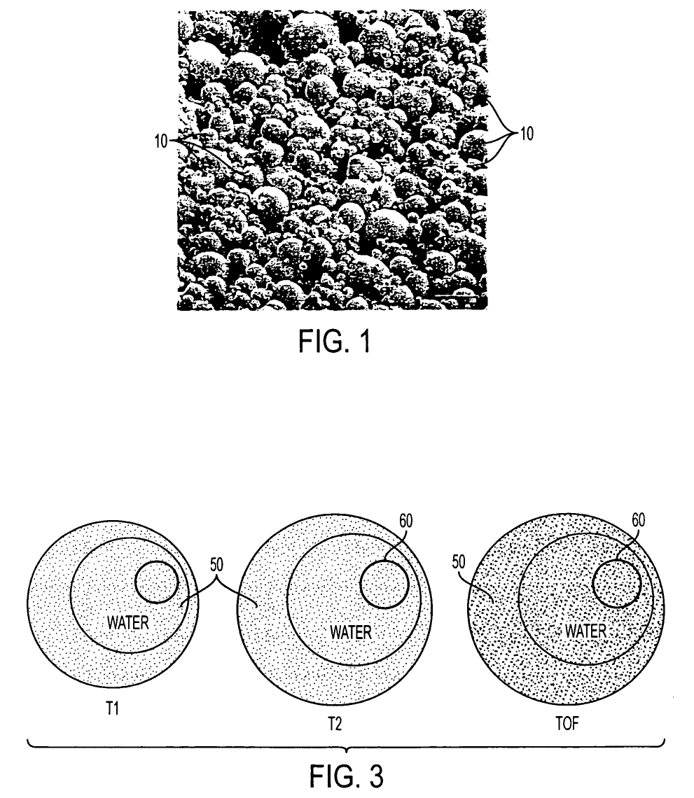

Referring now to FIG. 1, a multiplicity of stable, dry, fluid-based microcapsules 10 suitable for constructing a MRI-enhanced medical device of the present invention are described. The microcapsules preferably include a high ratio of fluid volume to shell volume and are stable during long-term storage. The resulting medical devices that include the microcapsules preferably have a commercially acceptable shelf-life (e.g., >12 months) and are safe for patient contact. Additionally, the microcapsules preferably are compatible with a variety of commonly used medical polymers, such as polyethylene, polyurethane, nylon, polyethylene terephthalate, etc., are broadly compatible with existing polymer processing methodologies, and do not adversely impact the flexibility and strength of polymer matrices into which the microcapsules are incorporated.

The microcapsules may be formed by any of a variety of techniques. In a preferred method, the microcapsules are created using a three-stage coacervation process in which a fluid droplet of the core material is formed, a shell or wall is then formed around the fluid droplet to encapsulate the core material, and the capsule is then isolated. While the process of coacervation is described in greater detail, the microcapsules may be formed using other known processes, such as including vapor phase deposition, fluid bed coating, macroemulsion, entrapment/matrix encapsulation, dispersion polymerization, interfacial polymerization, solvent phase separation, liposomal encapsulation, pan coating, alginate encapsulation.

In one embodiment, microcapsules 10 are formed using medical grade polyurethane as the shell material and mineral oil as the core material. When formed using a coacervation process, the microcapsules preferably have a diameter of between about 10-80 microns, and more preferably between about 15-20 microns, wherein the shells have a wall thickness of 1-5 microns, and more preferably 1-2 microns.

As is understood in the art of microencapsulation, coacervation is a phenomenon wherein a solution comprising a colloid in an appropriate solvent is reduced so that it separates into a pair of phases. One of the phases is high and the other is low with respect to colloid concentration. In a dispersed state, the high-colloid phase appears as amorphous liquid droplets that coalesce over time into a single clear homogeneous high-colloid liquid layer. This layer is deposited to produce the wall of the resultant capsules.

Coacervation may be initiated in a variety of ways, including by changes in temperature, pH, or by adding an additional substance. Microencapsulation occurs as the coacervate wets the suspended core droplets and coalesces into a continuous coating. Alternatively, microencapsulation may be accomplished using any of a number of known physical encapsulation processes, including, for example, spray coating, pan coating, fluid bed coating, annular jet, spinning disk, spray cooling, spray drying, and spray chilling.

Preferred coacervation processes for use in producing microcapsules suitable for the present invention result in the microcapsules having an impermeable hard shell, which chemically isolates the core material from the polymer matrix during manufacture of the interventional device. In addition, the microcapsule shells should be sufficiently robust to survive chemicals typically used in device manufacture, including organic solvents used in adhesives and urethane polymers.

In accordance with the principles of the present invention, core materials should be selected that enhance MRI visibility, i.e., that produce high signal strength and high contrast with surrounding tissue, that are chemically non-reactive with the microcapsule shell material, and that are safe for patient contact. Preferred core materials exhibit a high density of free protons, short T1 values and long T2 values. Generally, T1 is short for lipids and increases with decreasing molecular size. On the other hand, T2 is long for most liquids and increases with molecular size and decreased fluid mobility.

Lipids and oils are particularly well suited to meet the aforementioned preferred microcapsule characteristics since they typically contain a high density of free protons, and thus provide a strong signal strength under MRI. Lipids and oils also are compatible with encapsulation by coacervation, and generally are safe to humans. Suitable natural and synthetic oils suitable for use in the microcapsules of the present invention include, but are not limited to mineral oil, cod liver oil, terpene (vitamin E), and polyunsaturated fatty acids such as oleic acid.

Fluid mobility through the capsule shell and into the surrounding polymer matrix also should be limited. Accordingly, the core fluid optionally may include modifying agents that reduce migration, such as small molecular weight T1 reducing agents and relaxation modifying agents. Suitable relaxation modifying agents include iron oxide nano-particles and anchored gadolinium based T1 reducing agents.

As noted above, the microcapsules preferably are compatible with a broad range of polymers having different processing procedures, such as are commonly used in medical device construction. Suitable polymer materials include compliant polyurethane and silicon rubbers, as well as rigid, machinable liquid plastics and epoxies. Microcapsules 10 preferably are compatible with a variety of manufacturing processes conventionally used with such compliant and rigid polymer materials. These processes include, for compliant materials, extrusion, dip, lacquer and spray applications, solvent and thermoforming using mandrels and molds. Processes used for rigid materials may include blow, compression or injection molding, followed by machining to achieve desired form factors and tolerances. Intense heat and pressure may be generated by cast plastics, which also may require thermal annealing to reduce internal stress before final machining.

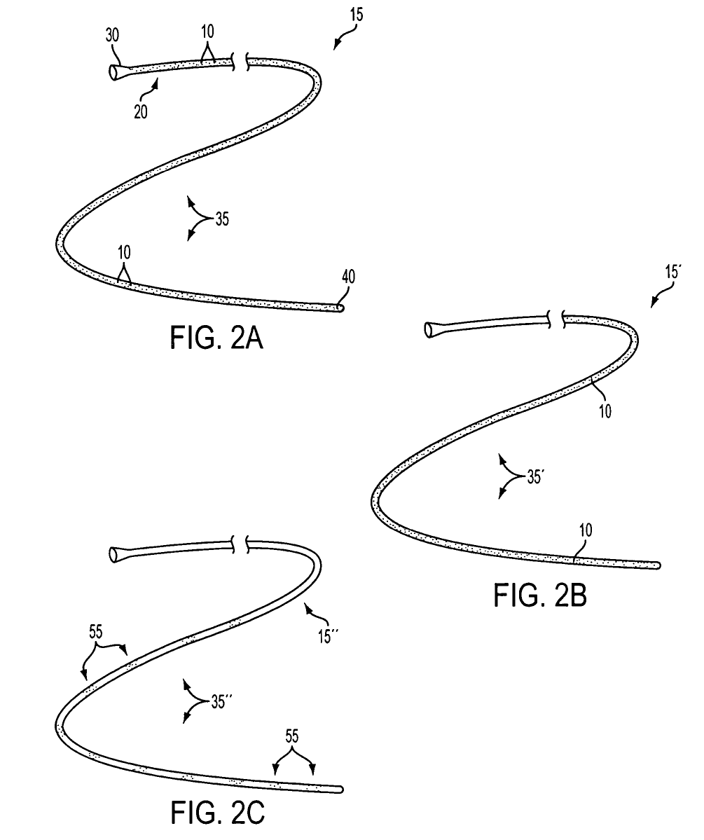

Referring now to FIG. 2A, exemplary MRI diagnostic catheter 15 in accordance with the present invention is described. Catheter 15 comprises an elongated tubular body formed from microcapsule enhanced medical grade polymers, and includes proximal end 20 with hub 30 and flexible distal end region 35 having working end 40. Microcapsules 10, such as described hereinabove, are incorporated in the polymer matrix of the catheter along the entire length of the catheter to provide the above-stated advantages of the present invention. Alternatively, a polymeric coating incorporating the microcapsules of the present invention may be applied to the exterior of an otherwise conventional interventional device.

With respect to FIG. 2B, an alternative embodiment of a catheter of the present invention is described, in which microcapsules 10 are incorporated only in distal end region 35′. Catheter 15′ may be manufactured using the components of a conventional vascular catheter (e.g., the SMART Peripheral Stent System manufactured by Cordis Corporation, Miami, Fla.), but in which the distal end region is replaced with a microcapsule-enhanced portion. Catheter 15′ therefore provides enhanced visualization of the catheter where it is typically most needed, and with little or no adverse impact on the mechanical properties or ease of use of the catheter compared to its conventional non-enhanced counterparts.

With respect to FIG. 2C, a further alternative catheter is described in which microcapsules 10 are disposed in multiple circumferential rings 55 that are axially spaced apart along distal end region 35″ of catheter 15″. In this embodiment, rings 55 may be assembled into a catheter of otherwise conventional construction using suitable thermal bonding methods or adhesives. Alternatively, rings 55 may be formed by applying short segments of heat-shrinkable tubing to a catheter of otherwise conventional construction.

An exemplary method of making a microcapsule-enhanced catheter in accordance with the principles of the present invention is now described. In an initial step, microcapsules 10, such as shown in FIG. 1, preferably having less than 1% microcapsule solids by weight, are thoroughly mixing with a high solid (e.g., 20-40%) solution of a medical grade polymer resin in organic solvent. The solution is then degassed until it breaks and settles. A guidewire is introduced into the degassed polymer solution, extracted at a steady pace and the coating is permitted to dry. Solidification typically takes place in approximately 5 minutes, and about 80% of end strength is attained during the first 24 hours after extraction.

In an alternative method of manufacture, microcapsule-enhanced medical devices may be formed using conventional extrusion equipment. In this case, microcapsules are mixed with granular polymeric material, e.g., polyethylene or polyurethane, heated, and then extruded through a die under pressure to form a continuous length of tubing having a predetermined diameter and wall thickness. Substantially uniform dispersion of the microcapsules may be achieved by completely mixing the microcapsules within the polymeric material at the desired weight and concentration.

Where it is desired to incorporate microcapsules only in a portion of a medical device, for example only in a distal region, the microcapsules may be periodically introduced into the polymeric material during the extrusion process, to form multiple circumferential microcapsule-enhanced rings axially spaced apart along the length of device, as depicted in the embodiment of FIG. 2C.

Referring now to FIG. 3, results of preliminary testing of an enhanced MRI-visible test samples constructed in accordance with the principles of the present invention are described. The test samples are similar in dimensions and construction to conventional catheters. To perform the test, microcapsule-enhanced and conventional samples were prepared for side-by-side MRI visibility testing. Sample 50 was constructed using a mixture of medical grade polyurethane and microcapsules (<1% microcapsule solids by weight) containing mineral oil. The mixture was processed using the methods described hereinabove to form a thin cylindrical test sample approximately 5 cm in length, 2 cm in diameter and 200 microns in thickness. Sample 60 was constructed using the same process with standard polyurethane materials to form a cylindrical test sample measuring 5 cm in length, 0.9 cm in diameter and 400 microns thick.

In general, smaller diameter, thicker samples are more easily visualized on MRI, so the test was biased to provide a distinct MRI visibility advantage to sample 60. This was done because it was expected that a standard polyurethane sample having the dimensions of the microcapsule-enhanced test sample would be been nearly invisible in the MRI image.

Both test samples were placed in saline fluid and imaged using a General Electric 1.5T whole body imager (System 5.8 with FSE and MRA option). The images were acquired using protocols previously established to characterize vessel wall layer characteristics. Three sets of images were acquired with different pulse sequences (T1, T2, TOF) to broadly test MRI visibility. In the accordance with the test protocol, the wall of the sample should appear bright in the MRI image if it exhibits MRI visibility.

As shown in FIG. 3, microcapsule-enhanced sample 50 exhibits increased MRI visibility for T1 and TOF pulse sequences as compared to the smaller diameter of sample 60 of standard construction. Results for T2 weighted images show a limited increase in MRI visibility for microcapsule-enhanced sample 50, but well above what would be expected for a standard catheter of similar dimensions. Accordingly, it is expected that through optimization of the core and shell materials, significantly enhanced MRI visibility may be obtained for medical devices, with little or no impact on the mechanical properties or ease of use of the devices.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

1. A medical device suitable for use with magnetic resonance imaging, the medical device comprising:
a polymeric component configured for attaining a diagnostic or therapeutic goal; and
a microcapsule additive disposed within at least a portion of the polymeric component, the microcapsule additive comprising a fluid impermeable shell component containing an MRI contrast-enhancing fluid.

2. The medical device of claim 1 wherein the polymeric component comprises a device body having a working end, the working end configured for performing a diagnostic or therapeutic procedure.

3. The medical device of claim 2 wherein the medical device is an interventional medical device and the device body comprises an elongated tubular body.

4. The medical device of claim 1, wherein the MRI contrast-enhancing fluid renders the portion of polymeric component directly visible during magnetic resonance imaging.

5. The medical device of claim 1, wherein the polymeric component comprises a contrast agent.

6. The medical device of claim 1, wherein the polymeric component comprises a coating.

7. The medical device of claim 1, wherein the MRI contrast-enhancing fluid exhibits a high density of free protons with clinically useful relaxation properties.

8. The medical device of claim 7, wherein the MRI contrast-enhancing fluid comprises a lipid or oil.

9. The medical device of claim 8, wherein the MRI contrast-enhancing fluid is selected from the group consisting of: mineral oil, cod liver oil, terpene and polyunsaturated fatty acids.

10. The medical device of claim 1, wherein the microcapsule additive is formed by coacervation.

11. The medical device of claim 1, wherein the microcapsule additive is formed by vapor phase deposition, fluid bed coating, entrapment/matrix encapsulation, macroemulsion, dispersion polymerization, interfacial polymerization, solvent phase separation, liposomal encapsulation or alginate encapsulation.

12. The medical device of claim 1, wherein the microcapsule additive is formed by spray coating, pan coating, fluid bed coating, annular jet, spinning disk, spray cooling, spray drying or spray chilling.

13. The medical device of claim 1, wherein the fluid impermeable shell component of the microcapsule additive is chemically compatible with the polymeric component.

14. The medical device of claim 1, wherein the polymeric component is selected from the group consisting of polyurethane, polyethylene, silicon rubber and polyethylene terephthalate.

15. The medical device of claim 1, wherein the MRI contrast-enhancing fluid further comprises relaxation modifying agents.

16. The medical device of claim 15, wherein the relaxation modifying agents are selected from the group consisting of iron oxide particles and anchored gadolinium-based T1 reducing agents.

17. An interventional device having enhanced visibility during magnetic resonance imaging, the interventional device comprising:
a device body comprising a polymeric matrix and having proximal and distal ends and a distal end region; and
a microcapsule additive disposed within the polymeric matrix in at least a portion of the distal end region, the microcapsule additive comprising an MRI contrast-enhancing fluid.

18. The interventional device of claim 17, wherein the MRI contrast-enhancing fluid comprises a lipid or oil.

19. The interventional device of claim 18, wherein the microcapsule additive further comprises a fluid impermeable shell component that is chemically compatible with the polymeric matrix.

20. The interventional device of claim 17, wherein the MRI contrast-enhancing fluid further comprises a relaxation modifying agent.

21. The interventional device of claim 17, wherein the interventional device is formed by extrusion of a mixture of the polymeric material and the microcapsule additive.

22. The interventional device of claim 17, wherein the interventional device is formed by coating the polymeric material onto the device body.
